(19) United States
(12) Patent Application Publication
(10) Pub. No.: US 2024/0390584 A1
(43) Pub. Date: Nov. 28, 2024

(54) Electronics Device with Battery Life Optimisation Provided for a drug delivery device or a drug delivery add-on device

(71) Applicant: Sanofi, PARIS (FR)

(72) Inventors: Brian Molyneux, Monton, Manchester (GB); Ronald Smith, Salir, Loule (PT)

(21) Appl. No.: 18/694,104

(22) PCT Filed: Sep. 22, 2022
(86) PCT No.: PCT/EP2022/076285

§ 371 Date: Mar. 21, 2024

(30) Foreign Application Priority Data
Sep. 24, 2021 (EP) 21315167.3

(51) Int. Cl. A61M 5/20; A61M 5/315

(52) U.S. Cl. CPC A61M 5/20; A61M 5/31545; A61M 2205/50; A61M 2205/52; A61M 2205/8212

(57) ABSTRACT

An electronics device provided for a drug delivery device or a drug delivery add-on device is disclosed, wherein the drug delivery device or the drug delivery add-on device comprises a dose measurement function and a data transmission function. The electronics device comprises at least one first controller unit being provided for controlling dose related functions of the drug delivery device or drug delivery add-on device and for data processing functions comprising one or more first numerical calculations with dose related data, and at least one second controller unit being provided for data processing functions comprising one or more second numerical calculations with dose related data having a higher complexity than the first numerical calculations and communication functions, wherein the at least one first controller unit is designed for using less power than the at least one second controller unit.

### FIELD

The present disclosure relates to an electronics device with battery life optimisation provided for a drug delivery device or a drug delivery add-on device.

### BACKGROUND

WO2010/052275A2 describes an electronic drug delivery device, which optionally can be equipped with means for transferring data with an external device, where the drug delivery device incorporates a power-management method which is effective in minimizing power consumption for the incorporated electronic circuitry yet allows ease of use during operation of the device. In an exemplary embodiment the device has a low-power hibernating state in which two functions, e.g., detection and communication means, are in a low-power sleep modus, a high-power state in which both of the functions, e.g., the detection and the communication means, are in an energized high-power state, and a medium-power state in which one function, e.g., the detection means, is in an energized high-power state and a second function, e.g., the communication means, are in a low-power sleep modus.

WO2020/257137A1 describes adding communication functionality to a drug delivery device for purposes of transferring information to a user device (e.g., a mobile computing device such as a smartphone, a personal computer, a server, etc.) while maintaining power-efficient operation. In one aspect, the drug delivery device comprises: a reservoir adapted to contain a drug, an injection mechanism coupled with the reservoir to deliver a drug from the reservoir, a power source, one or more sensors, a memory, a controller powered by the power source and having an active mode and a low-power mode. The controller is configured to, while operating in the active mode, use the one or more sensors to detect that the injection mechanism has performed an injection. The controller is also configured to generate in the memory a data entry indicative of the injection and/or a state of the drug delivery device, and switch into the low-power mode subsequent to or contemporaneous with detecting that the injection mechanism has performed the injection. The drug delivery device also comprises a wireless communication module powered by the power source and configured to establish a wireless connection with a user device while the controller is operating in the low power mode and transmit a message indicative of the injection and/or the state of the drug delivery device to the user device.

### SUMMARY

This disclosure describes electronics device with battery life optimisation provided for a drug delivery device or a drug delivery add-on device.

In one aspect the present disclosure provides an electronics device provided for a drug delivery device or a drug delivery add-on device, wherein the drug delivery device or the drug delivery add-on device comprises a dose measurement function and a data transmission function, and wherein the electronics device comprises at least one first controller unit being provided for controlling dose related functions of the drug delivery device or drug delivery add-on device and for data processing functions comprising one or more first numerical calculations with dose related data and at least one second controller unit being provided for data processing functions comprising one or more second numerical calculations with dose related data having a higher complexity than the first numerical calculations and communication functions and wherein the at least one first controller unit is designed for using less power than the at least one second controller unit. By providing at least two controller units with different power requirements, particularly different power consumptions, and assigning different functions to the controller units with different power requirements, particularly data processing functions comprising numerical calculations, the battery life of battery-powered drug delivery devices and drug delivery add-on devices can be optimized. This approach is particularly based on the thought that the battery life can be improved when multiple, particularly heterogeneous controller units are used to implement different functions. For example, in embodiments the at least one first controller unit may lack advanced mathematical functions and also multiplication and division functionality, but can perform binary left and right shifts, and/or may lack support for 23 bit numbers. In embodiments, the at least one second controller unit may be capable of higher complexity computation such a performing trigonometric functions.

In an embodiment, the at least one first controller unit may be configured for at least one of the following: controlling a dose recording sensor system provided for measuring doses selected and expelled with the drug delivery device; acquiring data from the dose recording sensor system; performing the one or more first numerical calculations with the acquired data; detecting transitions at a movable encoder by comparing analogue measurement values obtained as dose related data with one or more thresholds and counting detected transitions to add up to a measured dose. These functions can be performed by a controller unit with a low power consumption, particularly with a controller unit specifically designed to perform these functions with the lowest possible power consumption.

In a further embodiment, the at least one second controller unit may be configured for at least one of the following: performing the one or more second numerical calculations with the acquired data, which have a higher complexity than the first numerical calculations; performing data communication with an external data processing device, particularly transmitting the acquired data after the performed first and/or second numerical calculation to the external data processing device. These functions usually require more power than the functions performed by the first controller unit, and, thus, can be handled more power efficiently with the second controller unit, which may be specifically designed to perform these functions.

In a yet further embodiment, the at least one first controller unit may comprise a single dose capture and recording controller unit configured to control the dose recording sensor system provided for measuring doses selected and expelled with the drug delivery device, to acquire data from the dose recording sensor system, and to perform the one or more first numerical calculations with the acquired data.

In embodiments, the at least one second controller unit may comprise a main controller unit configured for computation, particularly for performing the one or more second numerical calculations with the acquired data, which have a higher complexity than the first numerical calculations, and a communication controller unit configured for performing communication tasks, particularly for performing the data communication with the external data processing device, particularly transmitting the acquired data after the performed first and/or second numerical calculation to the external data processing device.

In further embodiments, the electronics device may be configured to activate one or more of the controller units only on demand, wherein activating a controller unit comprises switching the controller unit into a first operational state comprising a first functionality of the controller unit, and wherein deactivating a controller unit comprises switching the controller unit into a second operational state comprising a second functionality of the controller unit, which is reduced over the first functionality in order to reduce power consumption of the controller unit.

In a yet further embodiment, the electronics device may comprise a controller activation unit being configured to process an input signal of the electronics device and to activate one or more of the first and/or second controller units depending on the processing of the input signal.

In an embodiment, the controller activation unit may be configured to receive and process one or more of the following as input signal: a signal indicating a dose selection and/or expulsion by the drug delivery device; a signal indicating a demand for synchronizing data via communication; a signal indicating a demand for establishing communications with an external computing device.

In a further embodiment, the controller activation unit may be configured to activate one of the controller units for processing the input signal and for determining the desired function based on the input signal processing, and to activate one or more from the controller units depending on the determined desired function and to deactivate the other controller units.

In a yet further embodiment, the controller activation unit may be configured for an activation of the at least one second controller unit after the at least one first controller unit was activated and all functions performed by the at least one first controller unit were completed, and a deactivation of the at least one first controller unit when the at least one second controller unit has obtained data from the at least one first controller unit.

In embodiments, the at least one first controller unit and the at least one second controller unit may be discrete units connected by a data bus and/or at least some of the controller units are implemented by a system-on-chip comprising multiple cores each implementing one or more of the controller units.

In further embodiments, the at least one second controller unit may comprise one or more of the following: a wireless interface, particularly a Bluetooth®, Wi-Fi™, ZigBee™, a Near Field Communication interface; a wired interface, particularly a serial communication bus interface such as I2C, USB.

In a still further embodiment, the one or more first and/or second numerical calculations may comprise one or more of the following: digital signal processing; determining data heuristics; cryptography processing for the data communication; checksum calculation;

binary left and right shifts; logic decisions, for example on whether a measured dose should be changed to an error code; multilplications; divisions; trigonometric functions; one or more higher order calculations on a dose time and/or battery voltage, particularly to optimise and package data for storage and transmission.

In yet further embodiments, the at least one first controller unit has less program space than the at least one second controller unit. For example, the at least one first controller unit may comprise a program space of 2 k RAM (Random Access Memory)

In another aspect the present disclosure provides a drug delivery device or a drug delivery add-on device comprising an electronics device as disclosed herein and a battery for supplying the electronics device with electrical energy.

In an embodiment, the battery may be a button cell having a capacity in milliampere hours selected to supply an electrical current sufficient to operate the electronics device over the usual utilization time of the drug delivery device without requiring a battery replacement during the usual utilization time.

### BRIEF DESCRIPTION OF THE FIGURES

FIG. 1 shows an embodiment of a drug delivery device comprising an integrated wireless data communication circuitry and external devices communicating with the drug delivery device;

FIG. 2 shows an embodiment of a system comprising a drug delivery device, a wireless data communication accessory, and external devices communicating with the wireless data communication accessory;

FIG. 3 shows a block diagram of an embodiment of an electronics device provided for a drug delivery device e.g., from FIG. 1 or a drug delivery add-on device as shown in FIG. 2.

### DETAILED DESCRIPTION OF SOME EMBODIMENTS

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. The concept underlying the embodiments of the present disclosure is generally applicable to any drug delivery device with a dual functionality requiring dose measurement by any means and resulting in transmission of this dose measurement by any means.

The functionality integrated within the electronics device in the below described injection devices with connectivity to external devices generally comprises the following aspects:
dose capture and recording,
processing of the measured dose information, particularly together with ancillary information such as battery voltage and any diagnostics,
transmission of the dose information, particularly with other ancillary information, via a communication channel particularly established by a radio-based communication connection.

Usually, the electronics device is powered by a battery contained in the injection device and/or in an add-on device for the injection device. The required characteristics of a controller unit comprised by the electronics device to enable the above aspects with regard to power requirements may be quite different as explained in the following:
Dose capture and recording: ideally uses the minimum electric current possible to support a very restricted set of core functionality, targeted at measuring physical phenomena over a relatively long period of time, preferably up to one minute in duration. This controller function has usually no requirement for complex data processing or communication.
Processing: provides expedient and efficient processing of the data, potentially at the expense of ultimate power efficiency, for a shorter period of time preferably less than several seconds. Such numerical calculations may include e.g., digital signal processing, data heuristics, cryptography for communication, checksum calculation and others. This controller function has no requirement for data communication.
Data transmission: provides efficient, reliable data communication, e.g., via Bluetooth, for a shorter period of time preferably less than several seconds. This controller function has no requirement for ultra-low electric current consumption over longer periods.

Based on this, the approach described in this disclosure acknowledges that the above three functionality aspects have differing requirements from a controller unit and therefore aims to improve the overall power consumption, and consequently to extend the battery life, of an electronics device provided for a drug delivery device and/or a drug delivery add-on device particularly through providing controller units suitable for performing the different functionality aspects with regard to a minimal power consumption or at the highest possible power efficiency. According to the approach described in this disclosure, several controller units are used, which are provided to handle different functionality aspects, wherein controller units with assigned functionality aspects having lower power requirement are designed for using less power than controller units with assigned functionality aspects having higher power requirements. Even if this approach requires more than one controller unit, it better allows to adapt the power consumption to the required functionality of a drug delivery device and/or drug delivery add-on device. Moreover, the controller units can be activated only upon demand, e.g., when a certain functionality must be performed and the controller unit, to which this functionality is assigned, is required, and controller units can be shut down, when their assigned functionality is not required, wherein shutting down particularly means herein a switching of a controller unit into an operational state, in which the functionality provided by the respective controller unit is reduced in order to reduce the power consumption of the respective unit, particularly to a negligible power consumption with regard to the battery life.

Before an embodiment of the electronics device is described in detail, embodiments of drug delivery devices and drug delivery add-on devices with connectivity to external devices are described in detail. The electronics device disclosed herein is particularly suited for integration into these devices since they usually use a one-time and often non-exchangeable battery, which should last over the entire lifetime of the device or at least a predefined utilization time of 1 year or even longer. The batteries employed in these devices are usually button cells having a capacity in milliampere hours selected to supply an electric current sufficient to operate the electronics device over the usual utilization time of the device, which may be the entire lifetime of the device, e.g., when the device is a disposable drug delivery device, or a predefined utilization time, e.g., when the device is a reusable drug delivery device.

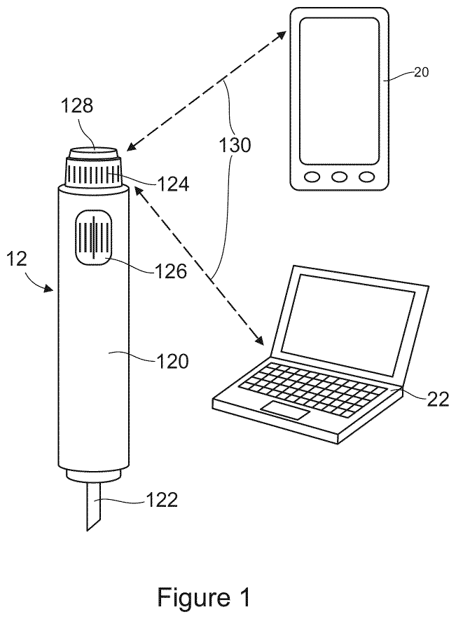

FIG. 1 shows a drug delivery device 12 in the shape of an insulin injection pen, e.g., the reusable pen injector as described in WO2014033195 or the disposable pen injector as described in WO2004078239. The device 12 comprises an elongated body 120 having a pen-like shaped form for holding a drug cartridge and a dose selection and delivery mechanism. At the lower end of the body 120, a syringe 122 for expelling a drug dose and injecting this dose in a patient's body is provided. The body 120 comprises at its other, upper, end a dial knob 124 for selecting a drug dose and an injection knob 128 for delivery of a selected dose. A user of the device 12 selects a dose by rotating the dial knob 124 around the longitudinal axis of the body 120. The selected dose is shown on a display 126 integrated in the body 120. After dose 25 selection, the user may press the injection knob 128 in the direction of the longitudinal axis for expelling the selected dose via the syringe 122 into a patient's body. The dose selection and delivery mechanism contained in the body may comprise electronics (not visible in FIG. 1) for detecting and for storing and transmitting selected and delivered doses.

Wireless data communication circuitry is integrated in the device 12, which may be part of an electronics device comprising wireless communication means for establishing a communication link 130 with an external device such as a smartphone 20 or a laptop computer 22, which may be paired with the wireless communication means. The term “paired” may mean that the wireless data communication circuitry and the external devices 20, 22 share some secret data such as cryptographic keys for establishing and/or securing data exchange.

The wireless data communication circuitry may be configured for establishing a long-range wireless communication link 130 via radio frequency communication, such as a Bluetooth® communication link and/or a Wi-Fi™ direct communication link based on the IEEE 802.11 standard (ISO/IEC 8802-11), with the external devices 20, 22 over a distance of at least several centimetres, particularly at least one meter, and more particularly several meters. The communication link 130 may be secured due to a pairing process initially made for enabling the communication.

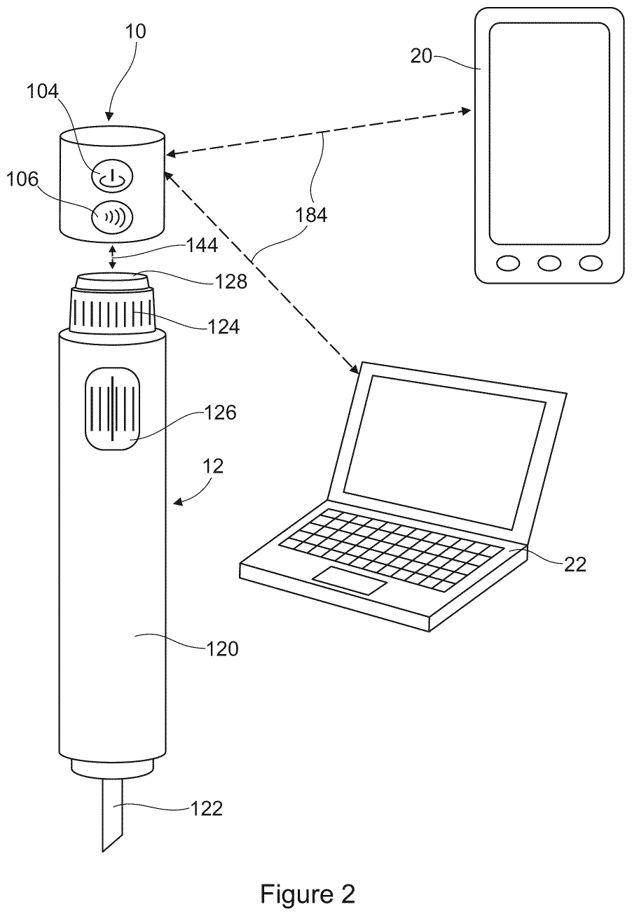

FIG. 2 shows a system comprising a drug delivery device 12 in the shape of an insulin injection pen, e.g., the reusable pen injector as described in WO2014033195 or the disposable pen injector as described in WO2004078239, a drug delivery add-on device in the form of a wireless data communication accessory 10 attachable to the device 12, and an external device such as a smartphone 20 or a laptop computer 22.

The wireless data communication accessory 10 can be attached to the device 12 by clipping it on the dial knob 124. The accessory 10 houses an electronics device (not shown) comprising a first wireless communication means for establishing a first communication link 184 with an external device such as a smartphone 20 or a laptop computer 22, which may be paired with the wireless communication means, and comprising a second wireless communication means and/or wired communication means for establishing a second communication link 144 for exchanging data with a data exchange interface of the device 12.

The accessory 10 may comprise a power button 104 for activating and deactivating the power supply of the electronics device manually. It may further comprise a wireless transmission button 106 provided for initiating a wireless transmission of data from the accessory to the external device 20 and/or 22. The button 106 may be also provided for initiating a pairing process of the accessory's 10 first wireless communication means with an external device 20, 22, e.g., by pressing the button 106 for a certain time period such as several seconds, thus switching the first wireless communication means into a pairing mode. The first wireless communication means can also be configured to establish automatically a communication link 184 with an already paired external device 20, 22 once the electronics device of the accessory 10 is powered and the external device 20, 22 is within the maximum communication range of the first wireless communication means.

In embodiments, activating the power supply of the electronics device of the accessory may be performed automatically, i.e., without manually pressing the button 104, e.g., by means of an integrated switch of the accessory, which may be activated when the accessory is attached to the device 12 or when a dose is selected and/or delivered with the device 12. The integrated switch may be, e.g., a mechanical switch or a magnetic switch, which may be activated when the accessory 10 is clipped on the dial knob 124 of the device 12 and/or when the dose selection and delivery mechanism is used by turning the dial knob 124 and/or pressing the injection knob 128.

The first communication means may be configured for establishing long-range wireless communications via radio frequency communication, such as a Bluetooth® communication link 184 and/or a Wi-Fi™ direct communication link based on the IEEE 802.11 standard (ISO/IEC 8802-11), with the external devices 20, 22 over a distance of at least several centimetres, particularly at least one meter, and more particularly several meters. The maximum distance provided for communication may depend on the power supply requirements of the accessory 10. E.g., when the accessory 10 is powered by a one-time usable battery, which should last several months, at least a year or even longer, the maximum distance may be configured by reducing the power requirements of the first communication means to meet the desired battery lifetime.

The second wireless communication means may be configured to establish short-range wireless communications employing electromagnetic induction for data transmission such as a short-range data communication technology based on an RFID (Radio Frequency Identification) standard such as NFC.

For securing communication via the first wireless communication link 184, cryptographic information assigned to the drug delivery device 12 may be used. The cryptographic information may comprise some secret data such as one or more cryptographic keys, e.g., a symmetric encryption key or a public-private-key pair for an asymmetric encryption.

A pairing of the accessory 10 and drug delivery device 12 may be required for data exchange. The pairing may, e.g., comprise receiving and storing some identification information from the drug delivery device 12 in a storage of the accessory 10, which, e.g., may be used to tag data received from the drug delivery device 12 and/or to ensure that only data from paired devices 12 are read by the accessory.

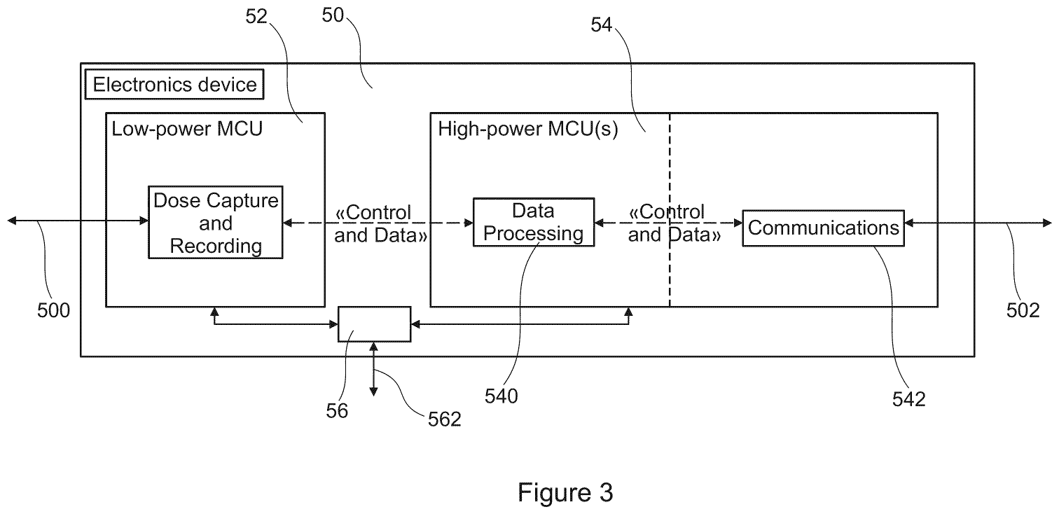

FIG. 3 shows a block diagram of an electronics device 50 provided for the drug delivery device 12 and/or the drug delivery add-on device 10. The electronics device 50 is designed to improve the battery life of the drug delivery device 12 and/or drug delivery add-on device 10 particularly by employing heterogeneous controller units, particularly microcontroller units (MCUs), provided for different functionality aspects and having different power requirements. E.g., one or more low-power controller units may be provided for longer duration dose measurement tasks and one or more higher-power controller units may be provided for shorter duration data processing and communication tasks, as shown in the block diagram of FIG. 3 and described below in more detail.

The electronics device 50 comprises at least one first controller unit 52, which may be implemented by a low-power MCU. The first controller unit 52 is provided for controlling dose related functions of the drug delivery device 12 and/or of the drug delivery add-on device 10, particularly dose capture and recording (double arrow 500). The first 25 controller unit 52 may be configured, e.g., by some dedicated controller firmware, for controlling a dose recording sensor system (not shown) of the drug delivery device 12 or the drug delivery add-on device 10. The first controller unit 52 may also comprise a single dose capture and recording controller unit, e.g., implemented by a further MCU, which may be configured to control the dose recording sensor system.

The dose recording sensor system may be provided to detect selection of a dose to be expelled via the syringe 122 into a patient's body, to detect expelling of a selected dose when the injection knob 128 is pressed, and to record an expelled dose into an internal memory, particularly together with further data such as date, time, drug identification, and/or patient identification. The dose recording sensor system may comprise one or more sensors integrated in the body 120 of the drug delivery device 12, particularly in the dose selection and delivery mechanism, in order to detect selection of a dose and/or expelling of a dose. E.g., the dose recording sensor system may comprise one or more magnetic sensors (e.g., Hall elements) and/or optical sensors as disclosed in WO2019/101962A1 for detecting a selected dose.

The first controller unit 52 may be also configured to perform one or more first numerical calculations with the acquired data. The so processed acquired data may then be stored in the internal memory for further processing by another controller unit. The first numerical calculations may comprise basic numerical calculations with a low complexity, which do not require much power and can be performed within a predefined power budget provided for the first controller unit 52 to perform its assigned tasks.

Generally, the first controller unit 52 is designed to consume the least power possible for the assigned tasks controlling a dose recording sensor system, acquiring data from the dose recording sensor system, and/or performing one or more first numerical calculations with the acquired data. This may be accomplished in that the first controller unit 52 only uses circuitry to perform the assigned tasks, such as e.g., a circuitry for receiving measurements from the dose recording sensor system, a circuitry for processing the received measurement, particularly including basic numerical calculations, and an internal storage for storing the processed measurements for further processing, and either does not comprise further circuitry or switches further circuitry, which is not required to perform the assigned tasks, off to save power.

For example, the first controller unit 52 may be configured for processing a received measurement from the dose recording sensor system in that it detects transitions at a movable encoder by comparing analogue measurement values received from the dose recording sensor system (dose related data) with one or more thresholds, particularly to determine a Gray code of the movable encoder, and counts detected transitions to add up to a measured dose.

The internal storage of the first controller unit 52 may be limited and have less program space than an internal storage of the second controller unit 54. For example, the internal storage of the first controller unit 52 may comprise 2 k program space, which would be suitable for implementing program code for performing the one or more first numerical calculations with the acquired data. Particularly, the first controller unit 52 may lack advanced mathematical functions and also multiplication and/or division due to the limited internal storage, but could do binary left and right shifts, i.e. some basic numerical calculations. Yet further, the first controller unit 52 may lack support for 32 bit numbers, which makes for example dealing with timestamps difficult.

The first controller unit 52 may be implemented e.g., by a standard microcontroller comprising the components required to perform the assigned tasks, an ASIC (Application Specific Integrated Circuit) or a PGA (Programmable Gate Array).

At least one second controller unit 54 is implemented in the electronics device 50 to perform tasks requiring more power than the tasks assigned to the first controller unit 52. The second controller unit 54 may be a high-power MCU, which may be designed for more complex tasks at the cost of requiring more power than the first controller unit 52.

The at least one second controller unit 54 may be configured to perform one or more second numerical calculations with the data acquired and processed by the first controller unit 52. E.g., the at least one second controller unit 54 may read from the internal memory of the first controller unit 52 stored data and process the read data by performing the second numerical calculations, which may have a higher complexity than the first numerical calculations. For example, the second controller unit 54 may be configured to perform numerical calculations such as multiplications, divisions, and even trogonmetric functions, and may be configured to support the processing of 32 bit numbers or even higher bit numbers.

For performing the one or more second numerical calculations, the second controller unit 54 may comprise a main controller unit 540 being provided for data processing and configured for performing the one or more second numerical calculation tasks, e.g., a dedicated numerical calculation logic, which is designed to perform the second numerical calculations under given constraints such as time and power requirements. The second controller unit 54 may comprise an internal storage, which may have more program space than an internal storage of the first controller unit 52. The larger program space of the second controller unit 54 may enable second numerical calculations with a computational complexity higher than the first numerical calculations.

The one or more first and/or second numerical calculations may comprise digital signal processing, e.g., a measurement signal received from the dose recording sensor system, determining data heuristics, e.g., with the data acquired from the dose recording sensor system, cryptographic processing for the data communication, e.g., encrypting data transmitted to an external device and decrypting encrypted data received from an external device, and/or checksum calculation, e.g., for detecting errors in a data transmission.

The at least one second controller unit 54 may further comprise a communication controller unit 542 being configured for performing data communication tasks, particularly for performing the data communication 502 with an external data processing device such as the smartphone or the laptop as shown in FIGS. 1 and 2. The communication controller unit 542 may comprise a wireless interface, particularly a Bluetooth®, Wi-Fi™, ZigBee™, a Near Field Communication interface and/or a wired interface, particularly a serial communication bus interface such as 12C or USB, for data exchange with an external device, particularly for transmitting the acquired data after the performed first and/or second numerical calculation to the external data processing device.

The second controller unit 54 may be implemented e.g., by a standard microcontroller comprising the components required to perform the assigned tasks, an ASIC (Application Specific Integrated Circuit) or a PGA (Programmable Gate Array).

The electronics device 50 may be further configured to activate the controller units 52, 54 particularly according to the following scheme:
1. By some kind of interaction, the electronics device 50 is activated, e.g., when a dose is selected or another user interaction with the drug delivery device and/or the drug delivery add-on device.
2. The second controller unit 54 is then activated to begin operation, to determine the type of operation required, e.g., measuring a dose, synchronize data via a communication, or pairing with an external device.
3. If dose capturing and recording is required, the first controller unit 52 assigned to dose capture and recording functionality is activated, and the second controller unit 54 may be deactivated so that power consumption during the dose capturing and recording phase may be minimised.
4. When dose capturing and recording is finished, as determined e.g., by the first controller unit 52 or a timer or other similar mechanism, the main controller unit 540 of the second controller unit 54 is activated, and data is retrieved from the first controller unit 52 before the latter controller unit is deactivated.
5. The main controller unit 540 then determines if communications functionality is required. If so, the communication controller unit 542 may be activated and communications may proceed.
6. Once communications have completed, the communication controller unit 542 is deactivated.
7. If no further functionality is required, the main controller unit 540 may deactivate itself until subsequent activation by a user.
8. At any point of the above activation sequence, if the dose capturing and recording functionality is required and is deemed to take precedence over data processing and communications functionality, then the main controller unit 540 may restart the sequence at step 1.

The above scheme is only an example, and the sequence can, e.g., be modified when required or when more controller units than one low-power and one high-power MCU are employed. Particularly with the above-described activation scheme, the power requirements of the electronics device may be minimized through judicious use of the appropriate controller units, particularly MCUs, during the various phases of operation.

Another relatively simple and fixed scheme may comprise that the first controller unit 52 may be activated by a dedicated signal, e.g., generated by a user interaction with the drug delivery device or the drug delivery add-on device, in order to provide the dose acquiring and recording functionality. The first controller unit 52 may be configured to output a signal, when its tasks are completed, thus, activating the second controller unit 54, which then can activate the main controller unit 540 to retrieve data from the internal memory of the low-power MCU 52 and to deactivate the first controller unit 52 after having retrieved all data. After processing of the data by the main controller unit 540, the second controller unit 54 may deactivate the main controller unit 540, and activate the communication controller unit 542, which prepares the processed data for transmission to an external device, establishes a communication connection with the external device, e.g., a Bluetooth® or Wi-Fi direct communication link, and transmits the processed data via the established communication connection.

For handling the activation of the controller units 52, 54, the electronics device May comprise a controller activation unit 56, which may be configured to process an input signal 562 and to activate the controller units 52, 54 depending on the processing of the input signal 562. The input signal 562 may comprise a signal indicating a dose selection and/or expulsion by the drug delivery device, e.g., a dose selection by turning the dial knob 124 (FIGS. 1, 2), a signal indicating a demand for synchronizing data via communication, e.g., a signal generated by the main controller unit 540 when the data processing is terminated or a timer signal, which is output a predefined timespan after termination of data acquisition and recording, and/or a signal indicating a demand for establishing communication with an external computing device, e.g., pressing of the wireless transmission button 106 of the drug delivery add-on device 10 from FIG. 2.

The controller activation unit 56 may be further configured to activate one of the controller units 52, 54 for processing the input signal, e.g., the controller unit 52. The activated controller unit such the unit 52 may then process the input signal 562 received from the controller activation unit 56 to determine the desired function, e.g., dose capturing and recording, a data synchronization request, or a communication establishment request with an external computing device. The so determined desired function may then be signalled to the controller activation unit 56, which may then activate all controller units required for performing the determined desired function and deactivate the other controller units, which are not required for performing the determined desired function.

Particularly, the controller activation unit 56 may be configured to implement a predefined activation scheme, such as an activation of the at least one second controller unit 54 after the at least one first controller unit 52 was activated and all functions performed by the at least one first controller unit 52 were completed, and a deactivation of the at least one first controller unit 52 when the at least one second controller unit 54 has obtained data from the at least one first controller unit 52. Thus, the controller activation unit 56 may in principle only receive a start signal 562 for initiating the activation scheme. The start signal 562 may be, e.g., generated by a sensor integrated in the drug delivery device or drug delivery add-on device, e.g., a movement detection sensor or a sensor provided for detecting a dose selection or even a button switch, which is pressed by a user when using the drug delivery device or the drug delivery add-on device.

The above-mentioned activation and deactivation of a controller unit may comprise switching a controller unit into a first operational state (activating) and a second operational state (deactivating). The first operational state may comprise a first functionality of the controller unit, particularly a functionality provided for fulfilling the assigned tasks by the controller unit. The second operational state may comprise a second functionality of the controller unit, which is reduced over the first functionality in order to reduce power consumption of the controller unit, e.g., it may comprise a sleep state, an ultra-low-power state, or another state, in which only a minimum functionality of the controller unit is maintained, and a minimum power consumption can be achieved.

The herein described approach of using heterogeneous controller units, particularly heterogeneous MCUs, with different capabilities and power requirements, particularly power usage, for performing tasks of an assigned functionality, enabling only the controller unit(s) that is (are) required for the functionality required at that moment in time, such as dose capture and recording, data processing or data transmission, enables the power consumption to be minimized and battery life extended and potentially optimized.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. E.g., in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be, or may include, a dual-chamber cartridge configured to store two or more components of the pharmaceutical formulation to be administered (e.g., an API and a diluent, or two different drugs) separately, one in each chamber. In such instances, the two chambers of the dual-chamber cartridge may be configured to allow mixing between the two or more components prior to and/or during dispensing into the human or animal body. E.g., the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, e.g., without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, e.g., that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, e.g., that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable amino acids, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, e.g., B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N—(ω-carboxyheptadecanoyl) human insulin.

Examples of GLP-1, GLP-1 analogues and GLP-1 receptor agonists are, e.g., Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, e.g., mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. E.g., the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present invention include, e.g., Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are e.g., acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system May involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1. An electronics device (50) provided for a drug delivery device (12) or a drug delivery add-on device (10), wherein the drug delivery device (12) or the drug delivery add-on device (10) comprises a dose measurement function and a data transmission function, and wherein the electronics device (50) comprises
at least one first controller unit (52) being provided for controlling dose related functions of the drug delivery device or drug delivery add-on device and for data processing functions comprising one or more first numerical calculations with dose related data and
at least one second controller unit (54) being provided for data processing functions comprising one or more second numerical calculations with dose related data having a higher complexity than the first numerical calculations and communication functions and
wherein the at least one first controller unit (52) is designed for using less power than the at least one second controller unit (54).

2. The electronics device (50) of claim 1, wherein the at least one first controller unit (52) is configured for at least one of the following:
controlling a dose recording sensor system provided for measuring doses selected and expelled with the drug delivery device (12);
acquiring data from the dose recording sensor system;
performing the one or more first numerical calculations with the acquired data;
detecting transitions at a movable encoder by comparing analogue measurement values obtained as dose related data with one or more thresholds and counting detected transitions to add up to a measured dose.

3. The electronics device (50) of claim 2, wherein the at least one second controller unit (54) is configured for at least one of the following:
performing the one or more second numerical calculations with the acquired data, which have a higher complexity than the first numerical calculations;
performing data communication with an external data processing device, particularly transmitting the acquired data after the performed first and/or second numerical calculation to the external data processing device.

4. The electronics device (50) of any of the claims 2 to 3, wherein the at least one first controller unit (52) comprises a single dose capture and recording controller unit configured to control the dose recording sensor system provided for measuring doses selected and expelled with the drug delivery device, to acquire data from the dose recording sensor system, and to perform the one or more first numerical calculations with the acquired data.

5. The electronics device (50) of any of the preceding claims, wherein the at least one second controller unit (54) comprises a main controller unit (540) configured for computation, particularly for performing the one or more second numerical calculations with the acquired data, which have a higher complexity than the first numerical calculations, and a communication controller unit (542) configured for performing communication tasks, particularly for performing the data communication with the external data processing device (20, 22), particularly transmitting the acquired data after the performed first and/or second numerical calculation to the external data processing device (20, 22).

6. The electronics device (50) of any of the preceding claims, being configured to activate one or more of the controller units (52, 54) only on demand, wherein activating a controller unit (52, 54) comprises switching the controller unit (52, 54) into a first operational state comprising a first functionality of the controller unit (52, 54), and wherein deactivating a controller unit (52, 54) comprises switching the controller unit (52, 54) into a second operational state comprising a second functionality of the controller unit (52, 54), which is reduced over the first functionality in order to reduce power consumption of the controller unit (52, 54).

7. The electronics device (50) of claim 6, comprising a controller activation unit (56) being configured to process an input signal of the electronics device (50) and to activate one or more of the first and/or second controller units (52, 54) depending on the processing of the input signal.

8. The electronics device (50) of claim 7, wherein the controller activation unit (56) is configured to receive and process one or more of the following as input signal: a signal indicating a dose selection and/or expulsion by the drug delivery device (12); a signal indicating a demand for synchronizing data via communication; a signal indicating a demand for establishing communications with an external computing device (20, 22).

9. The electronics device (50) of claim 8, wherein the controller activation unit (56) is configured to activate one of the controller units (52, 54) for processing the input signal and for determining the desired function based on the input signal processing, and to activate one or more from the controller units (52, 54) depending on the determined desired function and to deactivate the other controller units (52, 54).

10. The electronics device (50) of claim 9, wherein the controller activation unit (56) is configured for
an activation of the at least one second controller unit (54) after the at least one first controller unit (52) was activated and all functions performed by the at least one first controller unit (52) were completed and
a deactivation of the at least one first controller unit (52) when the at least one second controller unit (54) has obtained data from the at least one first controller unit (52).

11. The electronics device (50) of any of the preceding claims, wherein the at least one first controller unit (52) and the at least one second controller unit (54) are discrete units connected by a data bus and/or at least some of the controller units (52, 54) are implemented by a system-on-chip comprising multiple cores each implementing one or more of the controller units (52, 54).

12. The electronics device (50) of any of the preceding claims, wherein the at least one second controller unit comprises one or more of the following: a wireless interface (542), particularly a Bluetooth®, Wi-Fi™, ZigBee™, a Near Field Communication interface; a wired interface, particularly a serial communication bus interface such as I2C, USB.

13. The electronics device (50) of any of the preceding claims, wherein the one or more first and/or second numerical calculations comprise one or more of the following:
digital signal processing;
determining data heuristics;
cryptography processing for the data communication;
checksum calculation;
binary left and right shifts;
logic decisions;
multilplications;
divisions;
trigonometric functions;
one or more higher order calculations on a dose time and/or battery voltage.

14. The electronics device (50) of any of the preceding claims, wherein the at least one first controller unit has less program space than the at least one second controller unit.

15. A drug delivery device (12) or a drug delivery add-on device (10) comprising an electronics device (50) of any of the preceding claims and a battery for supplying the electronics device (50) with electrical energy, wherein particularly the battery is a button cell having a capacity in milliampere hours selected to supply an electrical current sufficient to operate the electronics device (50) over the usual utilization time of the drug delivery device (12) without requiring a battery replacement during the usual utilization time.
